(19) United States
(12) Patent Application Publication
(10) Pub. No.: US 2025/0195615 A1
(43) Pub. Date: Jun. 19, 2025

(54) METHODS AND COMPOSITIONS FOR THE TREATMENT OF ALCOHOL TOXICITY

(71) Applicant: The Board of Regents of The University of Texas System, Austin, TX (US)

(72) Inventors: David J. Mangelsdorf, Dallas, TX (US); Steven A. Kliewer, Dallas, TX (US); Mihwa Choi, Dallas, TX (US)

(21) Appl. No.: 18/845,140

(22) PCT Filed: Mar. 9, 2023
(86) PCT No.: PCT/US2023/064048

§ 371 Date: Sep. 9, 2024

Related U.S. Application Data
(60) Provisional application No. 63/319,073, filed on Mar. 11, 2022.

(51) Int. Cl. A61K 38/18

(52) U.S. Cl. CPC A61K 38/1825

(57) ABSTRACT

Provided herein are methods for treating acute alcohol toxicity using an agonist for a Fibroblast Growth Factor Receptor (FGFR) and βKlotho complex such as Fibroblast Growth Factor 21 (GFF21). Also provided are compositions and kits comprising these agonists (e.g., FGF21 (for administration to a subject in need.

### CROSS REFERENCE TO RELATED APPLICATIONS

This application claims the benefit of the U.S. Provisional application No. 63/319,073 filed Mar. 11, 2022, the disclosures of which is herein incorporated by reference in its entirety.

### ACKNOWLEDGEMENT OF GOVERNMENT SUPPORT

This invention was made with Government support under Grant No. AA028473 awarded by the National Institutes of Health (NIH). The government has certain rights in this invention.

### BACKGROUND

### 1. Field

The present disclosure is generally directed to compositions and methods for treating alcohol toxicity.

### 2. Discussion of Related Art

Acute alcohol poisoning is a potentially life-threatening consequence of consuming toxic amounts of ethanol in a short period of time. It can result in disorientation, loss of consciousness, impaired breathing and heart function, and even death. Emergency treatment for alcohol poisoning includes keeping the intoxicated individual upright and awake, if possible. Thus, drugs with sobering or “amethystic” (anti-intoxicant) activity could prove valuable for treating this dangerous condition.

Norepinephrine (NE) is an important neuromodulator in the CNS. Most central NE is synthesized in the locus coeruleus (LC), a small nucleus in the pons of the brainstem. LC neurons project extensively throughout the brain to regulate diverse biological processes, including arousal and alertness (Berridge et al., 2012; Poe et al., 2020). Fibroblast growth factor 21 (FGF21) is a hormone that is produced by the liver in response to a variety of metabolic stresses including starvation, protein deficiency, simple sugars and ethanol. It acts on a cell surface receptor composed of a conventional FGF receptor tyrosine kinase, FGFR1c, in complex with the single-pass transmembrane protein, β-Klotho (KLB). FGF21 binds directly to both FGFR1c and KLB, with FGFR1c serving as the effector of downstream signaling.

The present disclosure is based, at least in part, on the surprising discovery FGF21 can activate the noradrenergic pathway and exert sobering or “amethystic” effects on both arousal and motor coordination without changing ethanol catabolism. Accordingly, the disclosure herein demonstrates that the noradrenergic pathway may be targeted pharmaceutically to treat acute alcohol poisoning.

### BRIEF SUMMARY

In accordance with an aspect of the disclosure, provided herein are methods for treating acute alcohol intoxication in a subject in need thereof, the method comprising administering an effective amount of an agonist of a fibroblast growth factor receptor (FGFR)/β-klotho complex to the subject. In various aspects, treating acute alcohol intoxication can comprise reducing disorientation, increasing consciousness, improving heart function, improving respiration, or any combination thereof.

Also provided herein is a method for preventing acute alcohol intoxication in a subject in need thereof, the method comprising administering an effective amount of an agonist of a fibroblast growth factor receptor (FGFR)/β-klotho complex to the subject. In various embodiments, preventing acute alcohol intoxication can comprise preventing disorientation, preventing or averting unconsciousness, preventing depressed heart function or depressed respiration, or any combination thereof.

In any of the methods herein, the agonist of the FGFR/β-klotho complex may comprise FGF21 or an FGF21 congener. In various aspects, the effective amount of the agonist is administered as a pharmaceutical composition. In various aspects, the effective amount of the FGFR/β-klotho agonist (e.g., FGF21 or an FGF21 congener) may be from about 0.5 mg/kg to about 2 mg/kg, but may vary depending on the identity of the FGF21 congener which may be more or less potent than native FGF21.

In any of the methods of treating acute alcohol intoxication described herein, the subject may have imbibed alcohol less than an hour (e.g., within 10 minutes, within 20 minutes, within 30 minutes, within about 40 minutes, within about 50 minutes, or within about 60 minutes before the administration of the FGFR/β-klotho complex agonist (e.g., FGF21 or an FGF21 congener). In any of the methods of treating acute alcohol intoxication described herein, the subject may have imbibed alcohol between about 1 to 24 hours, between about 1 to 18 hours, between about 1 to 12 hours, between about 1 to 10 hours, between about 1 to 8 hours, between about 1 to 6 hours, between about 1 to 5 hours, or between about 1 to 4 hours before the administration of the FGFR/β-klotho complex agonist (e.g., FGF21 or an FGF21 congener).

In any of the methods of preventing acute alcohol intoxication described herein, the subject may imbibe alcohol less than an hour (e.g., within 10 minutes, within 20 minutes, within 30 minutes, within about 40 minutes, within about 50 minutes, or within about 60 minutes after the administration of the FGFR/β-klotho complex agonist (e.g., FGF21 or an FGF21 congener). In any of the methods of preventing acute alcohol intoxication described herein, the subject may imbibe alcohol between about 1 to 24 hours, between about 1 to 18 hours, between about 1 to 12 hours, between about 1 to 10 hours, between about 1 to 8 hours, between about 1 to 6 hours, between about 1 to 5 hours, or between about 1 to 4 hours after the administration of the FGF21.

In any of the methods provided herein, the subject may be a human.

Also provided are compositions comprising an agonist of a fibroblast growth factor receptor (FGFR) β-klotho complex and a pharmaceutically acceptable carrier or excipient. In some aspects, the agonist can comprise FGF21 or a congener thereof. The pharmaceutically acceptable carrier or excipient can comprise, in various embodiments, diluent, a binder, a filler, a buffering agent, a pH modifying agent, a disintegrant, a dispersant, a preservative, a lubricant or a combination of any thereof. In further aspects, the composition may be formulated for oral, nasal, or intravenous administration.

Still also provided are kits comprising an agonist of a fibroblast growth factor receptor (FGFR)/β-klotho complex, a pharmaceutically acceptable carrier or excipient, and instructions for administrating the activator to a subject suffering from acute alcohol intoxication. In various aspects, the agonist comprises FGF21 or a congener thereof. In some aspects, the kit further comprises a container and/or an administration device. In various aspects, the carrier or excipient included in the kit may comprise a diluent, a binder, a filler, a buffering agent, a pH modifying agent, a disintegrant, a dispersant, a preservative, a lubricant or a combination of any thereof. In various aspects, the subject may be a human.

### BRIEF DESCRIPTION OF THE DRAWINGS

Embodiments of the present disclosure are illustrated by way of example in which like reference numerals indicate similar elements and in which:

FIGS. 1A-1E. Fgf21−/− mice have a prolonged righting reflex recovery time after a binge ethanol dose. (A) Plasma FGF21 concentrations were measured in wild-type (WT) mice either 1 hour before administering ethanol (5 g/kg, oral gavage) or at the indicated times after ethanol (n=6/group). (B-E) WT and Fgf21−/− mice were administered ethanol (5 g/kg, oral gavage) and the following measurements made: time to loss of righting reflex (LORR) (B); LORR duration (C); plasma ethanol concentrations (D); brain ethanol concentrations (E) (n=5-6/group). All data represent the mean±SEM. *, P<0.05, **, P<0.01, ***, P<0.001 compared to −1 hour (A) or WT (C).

FIGS. 2A-2F. Hepatocyte-specific Fgf21−/− and neuron-specific Klb −/− mice have prolonged righting reflex recovery times after a binge ethanol dose. Control (Fgf21fl/fl) and hepatocyte-specific Fgf21-1-(Fgf21Alb) mice (A-C) or control (Klbfl/fl) and neuron-specific Klb −/− (KlbCamk2a) mice (D-F) were administered ethanol (5 g/kg, oral gavage) and the following measurements made: time to loss of righting reflex (LORR) (A, D); LORR duration (B, E); plasma ethanol concentrations (C, F) (n=5-7/group). All data represent the mean±SEM.

FIGS. 3A-3F. Pharmacologic FGF21 attenuates alcohol-induced sedation. (A, B) Wild-type (WT) male (A) or female (B) mice were administered ethanol (5 g/kg, oral gavage) followed 1 hour later by i.p. injection of vehicle or FGF21 at the indicated doses. Loss of righting reflex (LORR) duration was measured after FGF21 or vehicle administration (n=5-6/group). Data represent the mean±SEM. *, P<0.05, **, P<0.01, ***, P<0.001 compared to vehicle. (C, D) WT and Fgf21-1-mice (n=13-15/group) (C) or control (Klbfl/fl) and neuron-specific Klb −/− (KlbCamk2a) mice (n=12-16/group) (D) were administered ethanol (5 g/kg, oral gavage) followed 1 hour later by i.p. injection of FGF21 (1 mg/kg) or vehicle. LORR duration was measured after FGF21 or vehicle administration. Data represent the mean±SEM. Different lowercase letters indicate statistical significance (P<0.05). (E) WT mice were administered ethanol (2 g/kg, i.p.) followed 30 minutes later by i.p. injection of FGF21 (1 mg/kg; indicated by arrow) or vehicle. The time they could remain on a rotarod was measured, with 60 seconds the maximum (n=8/group). (F) WT and Fgf21-1-mice were administered ethanol (2 g/kg, i.p.) and the time on a rotarod measured as in (E). All data represent the mean±SEM.

FIGS. 4A-4D. Pharmacologic FGF21 does not attenuate other sedatives. Wild-type mice were administered ethanol (4.3 g/kg) (A) (n=9-12/group), ketamine (200 mg/kg) (B) (n=6/group), diazepam (30 mg/kg) (C) (n=5-6/group) or pentobarbital (55 mg/kg) (D) (n=7-8/group) by i.p. injection. After 30 minutes, mice were i.p. injected with either FGF21 (1 mg/kg) or vehicle. Loss of righting reflex (LORR) duration was measured after FGF21 or vehicle administration. All data represent the mean±SEM. *, P<0.05 compared to vehicle-treated mice.

FIGS. 5A-B. FGF21 is a physiologic regulator of noradrenergic neurons. (A) Representative confocal images of immunostaining for c-Fos and norepinephrine transporter (NET) in locus coeruleus sections prepared from wild-type (WT) and Fgf21-1-mice 2.5 hours after oral gavage with either water or ethanol (5 mg/kg). Scale bars represent 50 μM. (B) Quantification of c-Fos/NET co-expression (upper panel) and total NET-positive cell number (lower panel) (n=3 sections/mouse, 4 mice/group). Data represent the mean±SEM. Different lowercase letters indicate statistical significance (P<0.05). The drawing figures do not limit the present disclosure to the specific embodiments disclosed and described herein. The drawings are not necessarily to scale, emphasis instead being placed on clearly illustrating principles of certain embodiments of the present disclosure.

FIGS. 6A-D. Pharmacologic FGF21 activates norepinephrine neurons in the locus coeruleus. (A) Immunostaining was performed in locus coeruleus (LC) sections prepared from mice expressing TdTomato fused to the C-terminus of KLB. Antibodies against TdTomato and norepinephrine transporter (NET) were used. Top panels are confocal images. Bottom panels are composite Z-stack images. Scale bars represent 50 μM. (B and C) Immunostaining for c-Fos and NET in LC sections prepared from wild-type mice treated for 2 hours with vehicle or FGF21 (1 mg/kg, i.p.). Representative confocal images are shown in (B). Scale bars represent 50 μM. Quantification of c-Fos/NET co-expression (top panel) and total NET-positive cell number (lower panel) is shown in (C) (n=4 sections/mouse, 3 mice/group). Data represent the mean±SEM. ****, P<0.0001. (D, E) Immunostaining for c-Fos and NET in LC sections from groups of control (Klbfl/fl) and neuron-specific Klb −/− (KlbCamk2a) mice treated for 2 hours with vehicle or FGF21 as in (B) (n=3 mice/group). Quantification of c-Fos/NET co-expression (left panel) and total NET-positive cell number (right panel) is shown (E) (n=3 sections/mouse, 3 mice/group). Data represent the mean±SEM. Different lowercase letters indicate statistical significance (p<0.05).

FIGS. 7A-F. FGF21 exerts its amethystic activity through the noradrenergic nervous system. (A) Wild-type (WT) mice were injected with DSP-4 (50 mg/kg, i.p.) or vehicle. Two days later, the mice were administered ethanol (5 g/kg, oral gavage) followed 1 hour later by i.p. injection of FGF21 (1 mg/kg) or vehicle (n=16/group). Loss of righting reflex (LORR) duration was measured after FGF21 or vehicle administration. (B) WT mice were administered ethanol (5 g/kg, oral gavage) followed 1 hour later by i.p. injection of vehicle, FGF21 (1 mg/kg), prazosin (0.8 mg/kg) or FGF21+prazosin (n=8-10/group). LORR duration was measured after vehicle or FGF21 administration. (C) The experiment was performed as in (B) except mice were i.p. injected with vehicle, FGF21 (1 mg/kg), propranolol (10 mg/kg) or FGF21+propranolol (n=15-17/group). LORR duration was measured after vehicle or FGF21 administration. (D) Control (Klbfl/fl) and KlbDbh mice were administered ethanol (5 g/kg, oral gavage) followed 1 hour later by i.p. injection of vehicle or FGF21 (1 mg/kg). LORR duration was measured after FGF21 or vehicle administration (n=4-6/group). (E) Immunostaining for c-Fos and norepinephrine transporter (NET) was performed in locus (LC) coeruleus sections from groups of control (Klbfl/fl) and KlbDbh mice treated for 2 hours with vehicle or FGF21 (1 mg/kg) (n=3-4 mice/group). Quantification of c-Fos/NET co-expression (left panel) and total NET-positive cell number (right panel) are shown (n=3 sections/mouse, 3-4 mice/group). (F) Klbfl/fl mice bilaterally injected in the LC region with adeno-associated viruses (AAV) expressing either control (GFP) or Cre were administered ethanol (5 g/kg, oral gavage) followed 1 hour later by i.p. injection of vehicle or FGF21 at the indicated doses. LORR duration was measured after FGF21 or vehicle administration (n=8-11/group). All data represent the mean±SEM. Different lowercase letters indicate statistical significance (p<0.05).

FIG. 8. In situ hybridization of Klb (top panels) and Fgfr1c mRNA (middle panels) and the merge of the two (bottom panels). Two different magnifications are shown, with the boxed area in the left panels expanded in the right panels. The chromogenic signals for Klb and Fgfr1c were highlighted in green and red, respectively. Positions of the locus coeruleus (LC), lateral dorsal tegmental nucleus (LDTg) and ependymal cells (ep) adjacent to the fourth ventricle are indicated. Arrows indicate strong Klb signal.

### DETAILED DESCRIPTION

The instant disclosure is directed to compositions and methods of treating and/or preventing acute alcohol intoxication in a subject in need thereof. The methods described herein are based on the surprising discovery that the noradrenergic pathway can be modulated to an amythestic effect. As shown below, FGF21, a hormone that is induced in liver by starvation, protein deficiency, simple sugars and ethanol, can act on an FGF receptor/β-kotho complex to stimulate the noradrenergic system and reverse or alleviate alcohol intoxication. Accordingly, administration of an agonist for the FGF receptor/β-kotho complex (like FGF21 or congeners thereof) may be particularly useful in treating or preventing severe alcohol intoxication.

The following detailed description references the accompanying drawings that illustrate various embodiments of the present disclosure. The drawings and description are intended to describe aspects and embodiments of the present disclosure in sufficient detail to enable those skilled in the art to practice the present disclosure. Other components can be utilized, and changes can be made without departing from the scope of the present disclosure. The following description is, therefore, not to be taken in a limiting sense.

### 1. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Further, as the present disclosure is susceptible to embodiments of many different forms, it is intended that the present disclosure be considered as an example of the principles of the present disclosure and not intended to limit the present disclosure to the specific embodiments shown and described. Any one of the features of the present disclosure may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present disclosure may include a variety of combinations and/or integrations of the embodiments described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all value from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

In various embodiments, the subject is a mammal. For example, in various embodiments the subject is human.

Having described several embodiments, it will be recognized by those skilled in the art that various modifications, alternative constructions, and equivalents may be used without departing from the spirit of the present disclosure. Additionally, a number of well-known processes and elements have not been described in order to avoid unnecessarily obscuring the present disclosure. Accordingly, this description should not be taken as limiting the scope of the present disclosure.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### 2. Methods

Various aspects of the present disclosure provide methods for preventing or treating acute alcohol intoxication. The methods generally comprise administering an effective amount of an agonist for the FGFR/β-kotho complex to a subject in need thereof. In various embodiments, the agonist may comprise FGF21 or an FGF21 congener.

FGF21 functions through a receptor complex that consists of an FGF receptor (FGFR) and a co-receptor B-Klotho. Briefly, β-klotho is a transmembrane protein that interacts with the FGF receptor and is necessary to facilitate the binding of FGF21 and subsequent activation of the complex. As described further herein, this interaction between FGF21 and the FGF receptor/β-klotho complex results in a surprising amythestic effect that can be harnessed to treat or prevent alcohol poisoning/intoxication.

As used herein, an FGF21 congener is a substance, compound or moiety that acts as an agonist to the FGFR/β-klotho complex and therefore activates the noradrenergic system in a manner similar to or comparable to FGF21. In some aspects, the FGF21 congener can be a modified form of FGF21. For example, the FGF21 congener may be native FGF21 modified to increase or improve its pharmacokinetics (e.g., half-life). In some examples, the FGF21 congener may be an antibody or an antibody-like moiety that binds to the FGFR/β-klotho complex with high affinity and activates the complex (e.g., see Min et al., “Agonistic B-Klotho antibody mimics fibroblast growth factor 21 (FGF21) functions” J Biol Chem. 2018 Sep. 21; 293 (38): 14678-14688, which is incorporated herein by reference in its entirety). In some examples, the FGF21 congener may be a small chemical compound that can bind or modulate the FGFR/β-klotho complex. In other examples, the FGF21 congener may be a protein or peptide having an affinity for the FGFR/β-klotho complex.

In various aspects, the FGF21 congener may be one described in Taludar et al., (“FGF19 and FGF21: In NASH we trust” Molecular Mechanism Vol. 46, 2021, 101152) which is incorporated herein by reference in its entirety. For example, a suitable FGF21 congener to be used in the methods herein may be selected from the group consisting of LY2405319 (Eli Lilly), 05231023 (Pfizer), PF-05231023 (Pfizer), AKR-001 (Akero (AMG-876)), BMS-986036 (BMS), BKB8488A (Genentech), and NGM313 (MSD MK-3655).

Accordingly, in one aspect of the present disclosure, a method of treating acute alcohol intoxication in a subject in need thereof is provided. In various aspects, the method comprises activating the noradrenergic system in the subject. For example, the method can comprise activating the FGF21-NE pathway in a subject. In some embodiments, the method comprises administering an effective amount of a compound that acts on and activates the FGF21-NE pathway. In some aspects, the method comprises administering an effective amount of an agonist for a FGFR/β-kotho complex. In some aspects, the method comprising administering FGF21 or an FGF21 congener to the subject. In some embodiments, the method comprises administering an effective amount of FGF21 or an FGF21 congener to the subject. In some embodiments, an effective amount of FGF21 or an FGF21 congener is administered after a subject has imbibed alcohol. For example, in some embodiments, the subject imbibes alcohol between 1 and 24 hours, between 1 and 18 hours, between 1 and 12 hours, between 1 and 10 hours, between 1 and 8 hours, between 1 and 6 hours, between 1 and 5 hours, between 1 and 4 hours, between 1 and 3 hours, or between 1 and 2 hours before the administration of FGF21.

Also provided in a method of preventing acute alcohol intoxication and/or lessoning the effects of acute alcohol intoxication in a subject in need thereof, the method comprising administering an effective amount of an agonist to a FGF21 receptor/β-klotho complex (e.g., FGF21 or an FGF21 congener) to the subject prior to consumption of alcohol by the subject. In some embodiments, an effective amount of FGF21 is administered prior to imbibing alcohol. For example, in some embodiments, the subject imbibes alcohol between 1 and 4 hours, between 1 and 3 hours, or between 1 and 2 hours after administration of the agonist to an FGF21 receptor/β-klotho complex (e.g., FGF21).

In various aspects, the method of treating and/or preventing acute alcohol intoxication may comprise reducing disorientation, increasing consciousness, improving heart function, improving respiration, or any combination thereof.

In any of these embodiments, an effective amount of the agonist (e.g., FGF21 or congener thereof) may be about 0.5 mg/kg to about 10 mg/kg, from about 0.5 mg/kg to about 9 mg/kg, from about 0.5 mg/kg to about 8 mg/kg, from about 0.5 mg/kg to about 7 mg/kg, from about 0.5 mg/kg to about 6 mg/kg, from about 0.5 mg/kg to about 5 mg/kg, from about 0.5 mg/kg to about 4 mg/kg, from about 0.5 mg/kg to about 3 mg/kg or from about 0.5 mg/kg to about 2 mg/kg. For example, in some embodiments, the effective amount of the agonist (e.g., FGF21 or congener thereof) is about 0.5 mg/kg to about 2 mg/kg.

In any of these embodiments, an effective amount of the agonist may be administered to the subject in various doses. The doses may be delivered at a single time or over multiple days. In some aspects, an effective amount of the agonist comprises a dose of FGF21 or FGF21 congener of from about 1 to 100 mg. In some aspects, an effective dose may be from about 1 to 10 mg, from about 3 to 10 mg or from about 5 to 10 mg. For example, the effective dose may be about 3 mg, about 5 mg or about 10 mg. In various aspects, the effective dose may be from about 10 to 50 mg, from about 10 to 40 mg, from about 10 to 30 mg, from about 10 to about 20 mg, from about 20 to about 50 mg, from about 20 to about 40 mg, from about 20 to about 30 mg, from about 30 to about 50 mg, from about 30 to about 40 mg, or from about 40 to about 50 mg. For example, the effective dose may be about 20 mg, about 25 mg, about 28 mg or about 50 mg. In further aspects, an effective dose may be from about 50 mg to about 200 mg, from about 60 mg to about 200 mg, from about 70 mg to about 200 mg, from about 80 mg to about 200 mg, from about 90 mg to about 200 mg, from about 100 mg to about 200 mg, from about 110 mg to about 200 mg, from about 120 mg to about 200 mg, from about 130 mg to about 200 mg, from about 140 mg to about 200 mg, from about 150 mg to about 200 mg, from about 160 mg to about 200 mg, from about 170 mg to about 200 mg, from about 180 mg to about 200 mg, or from about 190 mg to about 200 mg. In some aspects, a suitable dosage is from about 50 mg to about 150 mg, from about 60 mg to about 150 mg, from about 70 mg to about 150 mg, from about 80 mg to about 150 mg, from about 90 mg to about 150 mg, from about 100 mg to about 150 mg, from about 110 mg to about 150 mg, from about 120 mg to about 150 mg, from about 130 mg to about 150 mg, from about 140 mg to about 150 mg. For example, in some aspects, the suitable dosage may be about 50 mg, about 70 mg, about 100 mg, about 140 mg, or about 150 mg.

In various embodiments, an effective amount of the agonist may depend on the identity of the agonist (e.g., whether FGF21 or a congener is used). As discussed above, a suitable FGF21 congener may be selected from LY2405319 (Eli Lilly), 05231023 (Pfizer), PF-05231023 (Pfizer), AKR-001 (Akero (AMG-876)), BMS-986036 (BMS), BKB8488A (Genentech), and NGM313 (MSD MK-3655). In

In any of the methods provided herein, the subject in need thereof may be a human.

### 3. Pharmaceutical Compositions

In any of the methods herein, the suitable agonist to an FGF21/β-Klotho complex (e.g., FGF21 or congener thereof) may be administered as a pharmaceutical composition.

FGF21 is a protein expressed by the FGF21 gene (NCBI Gene ID: 26291). Accordingly, it may be prepared for any of the pharmaceutical compositions or uses thereof provided herein according to standard techniques in the art. As an example, recombinant FGF21 may be prepared from transfected nucleic acids molecules using molecular biological methods known to in the art. In these methods, a polynucleotide sequence encoding the FGF21 is inserted into a vector that is able to express FGF21 when introduced into an appropriate host cell. Appropriate host cells are known to those of skill in the art and may include, but are not limited to, bacterial, yeast, insect and mammalian cells. Once expressed, FGF21 may be obtained from cells using common purification methods. For example, a secretion signal may be added to FGF21, allowing for secretion and isolation from cell culture supernatant. Alternatively, FGF21 lacking a secretion signal may be purified from inclusion bodies and/or cell extract. FGF21 may be isolated from culture supernatant, inclusion bodies or cell extract using any methods known to one of skill in the art, including for example, by chromatography (e.g., ion exchange, affinity, particularly by affinity for the specific antigen after Protein A, and sizing column chromatography), centrifugation, differential solubility, e.g. ammonium sulfate precipitation, or by any other standard technique for the purification of proteins; see, e.g., Scopes, “Protein Purification”, Springer Verlag, N.Y. (1982). Isolation of polypeptides is greatly aided when the polypeptide comprises a purification moiety.

In various aspects, the pharmaceutical compositions provided herein comprise an FGF21 congener (e.g., a compound, peptide, protein or moiety that activates the FGF/β-klotho complex). In some aspects, the congener may be an FGF21 modified to improve its pharmacokinetics or pharmaceutical usefulness. For example, the FGF21 may be modified to improve its half-life. Suitable modifications known in the art for this purpose include PEGylation, glycoPEGylation, modification of certain amino acid residues. Also envisioned are chimeric molecules comprising FGF21 or an FGF21 congener fused to an Fc domain. Also usable are bispecific agonist antibodies that bind the FGFR1 and KLB complex.

Accordingly, various aspects of the present disclosure are directed to pharmaceutical compositions, such as pharmaceutical compositions comprising FGF21 and a pharmaceutically suitable carrier or excipient.

The composition may comprise at least one excipient. Suitable excipients include pharmaceutically acceptable excipients, such as diluents, binders, fillers, buffering agents, pH modifying agents, disintegrants, dispersants, preservatives, lubricants, taste-masking agents, flavoring agents, coloring agents, or combinations thereof. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, or saccharides.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

The compositions disclosed herein may be formulated into various dosage forms and administered by a number of different means that will deliver a therapeutically effective amount of the active ingredient. The excipients included in the compositions comprising FGF21 may be based on the form of administering such compositions. Such compositions may be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term, “parenteral,” as used herein includes subcutaneous, intravenous, intramuscular, or intrasternal injection, or infusion techniques. In some embodiments, the pharmaceutical composition comprising FGF21 is formulated for nasal, intravenous or oral administration. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980).

### 3. Kits

Various aspects of the present disclosure are directed to kits for treating acute alcohol intoxication. In some aspects, the kits comprise FGF21, a pharmaceutically acceptable carrier or excipient, and instructions for administrating FGF21 to a subject suffering from acute alcohol intoxication.

In various embodiments, the kit comprises a pharmaceutical composition (e.g., any pharmaceutical composition provided herein) comprising FGF21 and the pharmaceutically acceptable carrier or excipient. In some embodiments, the kit further comprises a container. In some embodiments, the kit further comprises an administration device.

In various embodiments, instructions are included in the kit for administering FGF21 or a pharmaceutical composition comprising FGF21 to an individual suffering from acute alcohol intoxication.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1—General Methods

Mouse Studies All use of mice and related procedures were approved by the University of Texas Southwestern Medical Center's Institutional Animal Care and Use Committee. Experiments were performed with male mice unless indicated otherwise. Fgf21−/− (Potthoff et al., 2009), KlbCamk2a (Bookout et al., 2013), Fgf21Alb (Song et al., 2018) and Klb-TdTomato reporter mice (KLB-T) (Coate et al., 2017) have been described. KlbDbh mice were generated by crossing Dbh-Cre mice (Jackson Laboratory, Stock No: 033951) with Klbfl/fl mice (Bookout et al., 2013). Fgf21−/−, Fgf21Alb, and KLB-T mice were on a C57BL/6J background and KlbCamk2a and KlbDbh mice were on mixed C57BL/6J; 129/Sv backgrounds. C57BL/6J mice were generated by the University of Texas Southwestern Medical Center animal breeding core or purchased from Jackson Laboratory. Mice were housed in a temperature-controlled environment with 12 hour light/dark cycles and fed standard rodent chow ad libitum. All experiments were performed on age- and sex-matched mice. Recombinant human FGF21 protein was provided by Novo Nordisk and administered by i.p. injection at a dose of 1 mg/kg unless otherwise noted in the figure legends.

Viral Injections. Klbfl/fl mice were anesthetized using isoflurane anesthesia (3%-4% for induction; 1.5%-2% for maintenance) and positioned in a stereotaxic instrument (David Kopf Instruments) with a temperature controller to maintain body temperature. The skull was exposed, bregma was identified, and two small holes were drilled for bilateral injection into the LC (coordinates from lambda: +0.9 mm mediolateral, −0.9 mm antero-posterior and −3.82 mm dorso-ventral (Paxinos and Franklin, 2019)). Dorso-ventral coordinates are relative to pia. A volume of 300 nl (1×10¹² genomic particles/μl) of AAV8-GFP or AAV8-GFP-Cre virus (UNC Vector Core) was injected bilaterally into the LC. The virus was infused at 50 nl/minute using a microinjection syringe pump and Micro2T controller system with a 34G Nanofil needle (World Precision Instrument, UMP3T-1). After each injection, the needle was maintained in position for 10 minutes to prevent backflow and then slowly removed over 5 minutes. The skin was closed using sutures. Mice were allowed to recover for two weeks before use in experiments.

Loss of Righting Reflex (LORR) Studies. Time to LORR was defined as the time between ethanol administration and LORR. Once ataxic, mice were placed in a supine position in V-shaped plastic troughs and the time measured until they were able to right themselves three times within 30 seconds, which was defined as the duration of LORR. For studying FGF21 effects on ethanol-induced sedation, mice were administered ethanol (5 g/kg) by oral gavage followed 1 hour later by i.p. injection of either FGF21 or vehicle. For the anesthetics study, ketamine (200 mg/kg), diazepam (30 mg/kg), pentobarbital (55 mg/kg) or ethanol (4.3 g/kg) were i.p. injected followed 30 minutes later by i.p. injection of FGF21 or vehicle. Diazepam and ketamine were diluted in 0.9% saline and pentobarbital was dissolved in 0.9% saline containing 10% ethanol.

DSP-4, Prazosin and Propranolol studies. For the DSP-4 studies, C57BL/6J mice were i.p. injected with either DSP-4 (50 mg/kg) or vehicle. Two days later, mice were administered ethanol (5 g/kg) by oral gavage followed 1 hour later by i.p. injection of either FGF21 or vehicle. For the prazosin and propranolol studies, mice were administered ethanol by oral gavage followed 1 hour later by i.p. injection of FGF21 or vehicle in the presence or absence of prazosin (0.8 mg/kg) or propranolol (10 mg/kg). DSP-4, prazosin and propranolol were all dissolved in 0.9% saline just prior to use.

Rotarod Studies. Mice were trained on a Rotamex-5 rotarod (Columbus Instruments) at 5 rpm, with training complete when mice were able to stay on the rotarod for 60 seconds. To evaluate FGF21's effect on ethanol-impaired motor coordination, mice were i.p. injected with ethanol (2 g/kg) followed 1 hour later by i.p. injection of either FGF21 or vehicle. Time on the rotarod, with a maximum of 60 seconds, was measured at regular intervals.

c-Fos Induction and Immunohistochemistry. For the c-Fos induction studies, mice were habituated for four days by either i.p. injection of 0.9% saline or oral gavage with water. On the fifth day, mice were i.p. injected with vehicle or FGF21 (2 hour treatment), or orally gavaged with water or ethanol (5 g/kg; 2.5 hour treatment). Mice were anesthetized with isoflurane and transcardially perfused first with PBS followed by 10% neutral buffered formalin (NBF). Brains were fixed for 24 hours in 10% NBF at 4° C. and slices were prepared using a Leica VT1000S vibratome at a thickness of 50 μm. Brains from untreated KLB-T mice were processed in the same way. Slices were incubated for 1 hour in blocking buffer (1% bovine serum albumin, 5% normal goat serum, 0.3% Triton X-100 in PBS) at room temperature with shaking followed by incubation in primary antibodies, including antibodies against NET (Mab Technologies, 1:1000 dilution), cFos (Cell Signaling Technology, 1:1000 dilution) red fluorescent protein (Rockland, 1:500 dilution) and GFP (Aves Labs, 1:2000 dilution) for 48 hours at 4° C. Free-floating slices were washed 3 times in PBS for 10 minutes followed by incubation for 1 hour at room temperature with Alexa Fluor-conjugated secondary antibodies, including goat anti-mouse, goat anti-chicken, goat anti-rabbit IgGs (Invitrogen, 1:500 dilution), and DAPI (Fisher Scientific, 1:5000 dilution) in blocking buffer. Slices were washed 3 times for 10 minutes in PBS and mounted with Aqua-Poly/Mount (Polysciences). Images were taken using a Zeiss LSM780 confocal microscope and images were processed using ImageJ software. c-Fos counts were performed blinded.

In Situ Hybridization Analysis. C57BL/6J mice were anesthetized with isoflurane and transcardially perfused first with PBS followed by 10% neutral buffered formalin. Brains were fixed for 24 hours at 4° C. in 10% NBF. Brain slices were prepared using a Leica freezing microtome at a section thickness of 25 μm. In situ hybridization was performed using RNAscope multiplex fluorescence kits. Klb and Fgfric probes were purchased from Advanced Cell Diagnostics. Hybridized sections were counterstained with hematoxylin, dehydrated, cleared, and mounted with EcoMount (BioCare Medical). Images were taken using a Zeiss LSM780 confocal microscope.

FGF21 and Ethanol Measurements. For measuring FGF21 and ethanol concentrations in murine plasma, blood was centrifuged at 3,000 rpm for 15 minutes immediately after collection and plasma was stored at −80° C. until analysis. Plasma FGF21 concentrations were measured using the FGF21 mouse/rat ELISA kit (BioVendor) according to the manufacturer's instructions. Plasma ethanol concentrations were measured using the EnzyChrom ethanol assay kit (BioAssay Systems) according to the manufacturer's instructions. For measuring brain ethanol concentrations, brains were removed, frozen immediately in liquid nitrogen and stored at −80° C. Frozen whole brains were homogenized in 0.1N HCl and centrifuged at 13,000 rpm for 30 minutes at 4° C. Ethanol concentrations in the supernatants were measured using the EnzyChrom ethanol assay kit.

Statistical Analyses. All data are expressed as the mean±SEM. Statistical analyses were performed using GraphPad Prism Software Version 9.0. Unpaired two-tailed student's t tests were used for two group analyses. Multiple groups were tested by one-way or two-way ANOVAs with Tukey's multiple comparison test. For the rotarod analyses, a linear mixed effect model was fitted using the R package Ime4 and group effect P values derived using a likelihood-ratio test. In all analyses, a P value <0.05 was considered significant.

### Example 2-FGF21 Deficiency Exacerbates Ethanol-Induced Intoxication

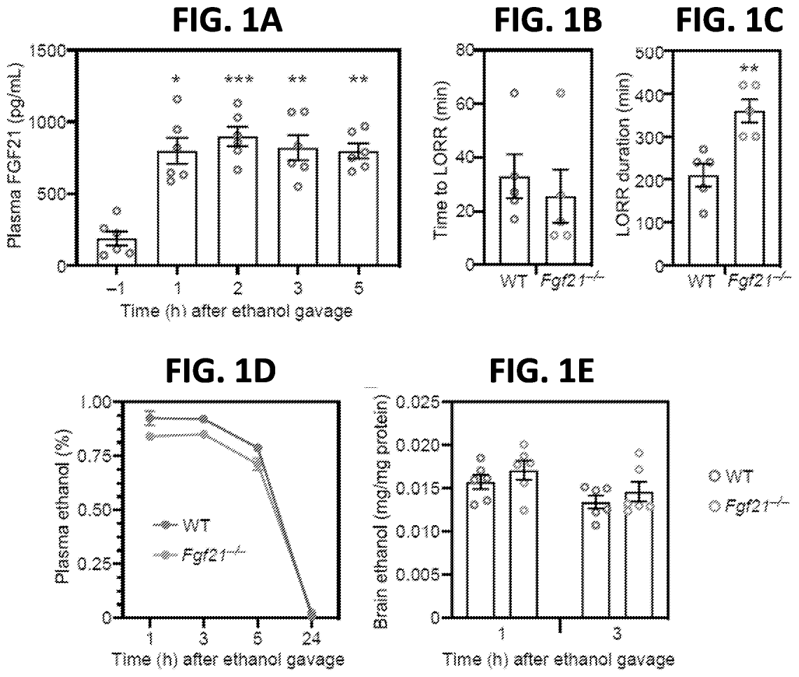

The effect of a single, binge dose of ethanol (5 g/kg by oral gavage) was compared between wild-type (WT) and global Fgf21−/− mice. As expected, plasma FGF21 was induced by ethanol in WT mice, peaking at 2 hours (FIG. 1A). In rodents, impaired righting reflex is a standard marker of inebriation. WT and Fgf21−/− mice lost their righting reflex at comparable times after ethanol gavage (FIG. 1B). Notably, however, Fgf21−/− mice required significantly more time to recover their righting reflex (FIG. 1C). WT and Fgf21−/− mice cleared ethanol from the plasma at the same rate (FIG. 1D), and brain ethanol concentrations were similar between the genotypes (FIG. 1E). Thus, FGF21 protects against ethanol-induced sedation without affecting ethanol catabolism

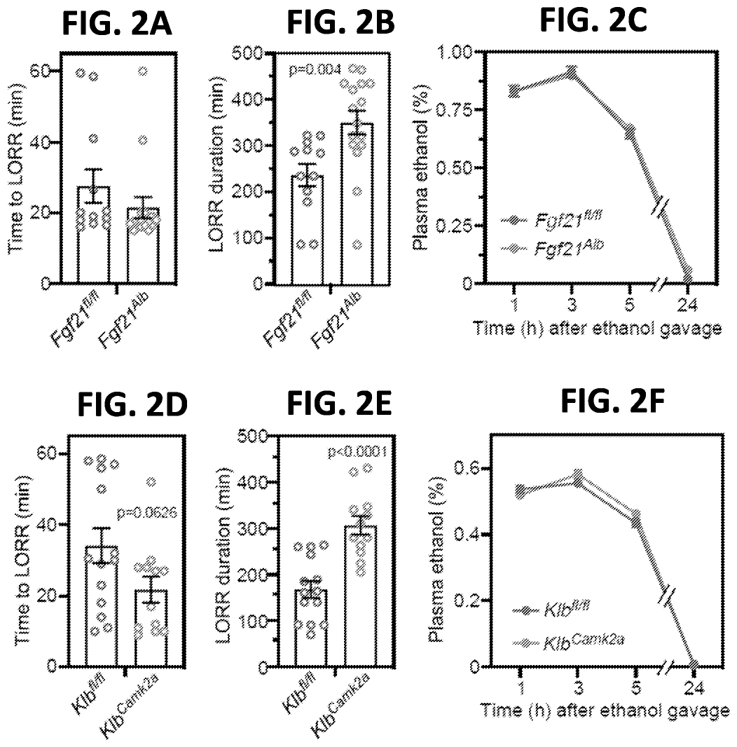

Similar results were obtained for both time to loss of righting reflex and its duration with hepatocyte-specific Fgf21-knockout (Fgf21Alb) mice (FIG. 2A and FIG. 2B) and neuron-specific Klb-knockout (KlbCamk2a) mice (FIG. 2D and FIG. 2E). There were no differences in plasma ethanol clearance between the knockout lines and control mice (FIG. 2C and FIG. 2F). These results indicate that liver-derived FGF21 attenuates ethanol-induced sedation by acting on the nervous system.

### Example 3-Pharmacologic FGF21 is Amethystic

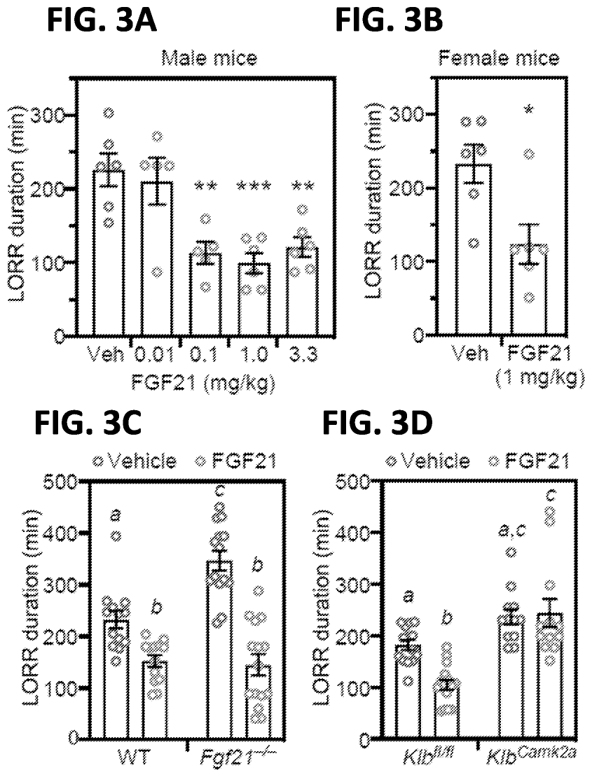

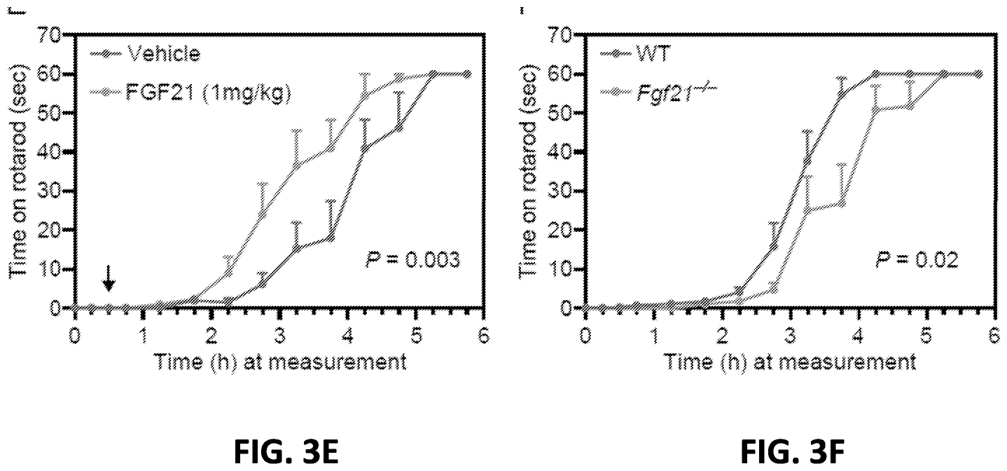

Pharmacologic FGF21 treatment was tested to decrease the time to righting reflex recovery after ethanol administration. WT mice were administered the binge ethanol dose by oral gavage followed by i.p. FGF21 injection one hour later. Remarkably, FGF21 administration reduced the time required for both male and female mice to recover their righting reflex by ˜90 minutes, reflecting a roughly 50% decrease (FIG. 3A and FIG. 3B). This effect was dose dependent and maximally efficacious at 1 mg/kg FGF21 in male mice (FIG. 3A).

The same pharmacologic FGF21 rescue experiment was performed in Fgf21−/− and KlbCamk2a mice. In Fgf21−/− mice, FGF21 administration reduced the righting reflex recovery time to that seen in WT mice (FIG. 3C). In contrast, FGF21 had no effect on righting reflex recovery time in KlbCamk2a mice (FIG. 3D), indicating that pharmacologic FGF21 exerts its amethystic effect by acting on the nervous system.

It was also investigated whether FGF21 inhibits ethanol-induced impairment of motor coordination. Pharmacologic FGF21 treatment reduced the time required for WT mice to recover their coordination on a rotarod following administration of a moderate dose of ethanol (2 g/kg, i.p.) (FIG. 3E). Conversely, recovery time was significantly increased in Fgf21−/− compared to WT mice (FIG. 3F). Thus, FGF21 is amethystic for ethanol-induced impairment of both righting reflex and motor coordination.

### Example 4-FGF21's Amethystic Activity is Selective for Ethanol

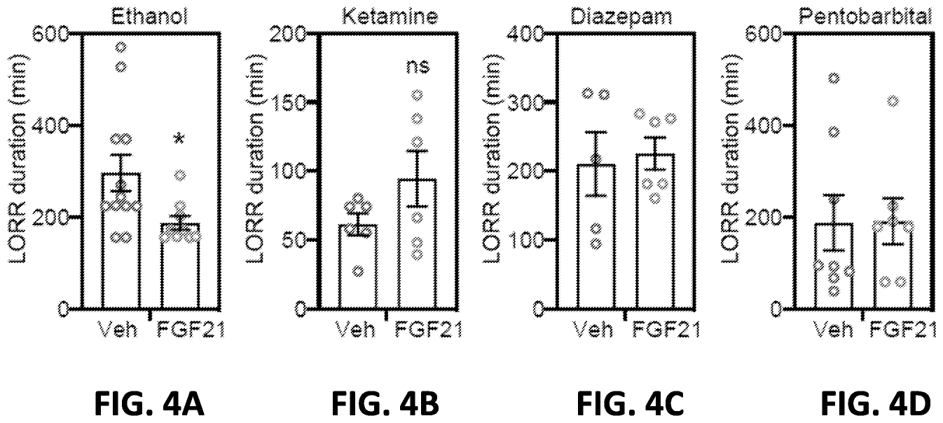

We tested whether FGF21 counteracts other sedatives that act through different mechanisms, including the glutamatergic receptor antagonist ketamine and the GABA receptor agonists diazepam and pentobarbital. Because these sedatives act more quickly and for a shorter duration than ethanol, we compressed the experimental timeline. FGF21 retained its ability to reduce righting reflex recovery time in ethanol-treated mice under these modified conditions (FIG. 4A). In contrast, FGF21 administration did not reduce the righting reflex recovery time for ketamine, diazepam and pentobarbital (FIG. 4B-4D). Thus, FGF21's amethystic activity is selective for ethanol.

### Example 5-FGF21 is a physiologic regulator of noradrenergic neurons

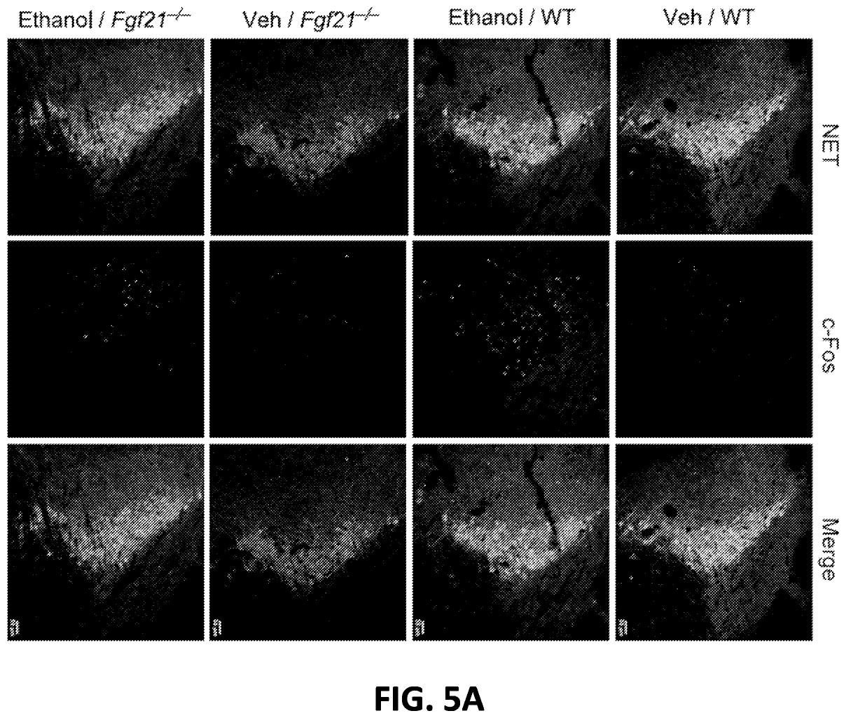

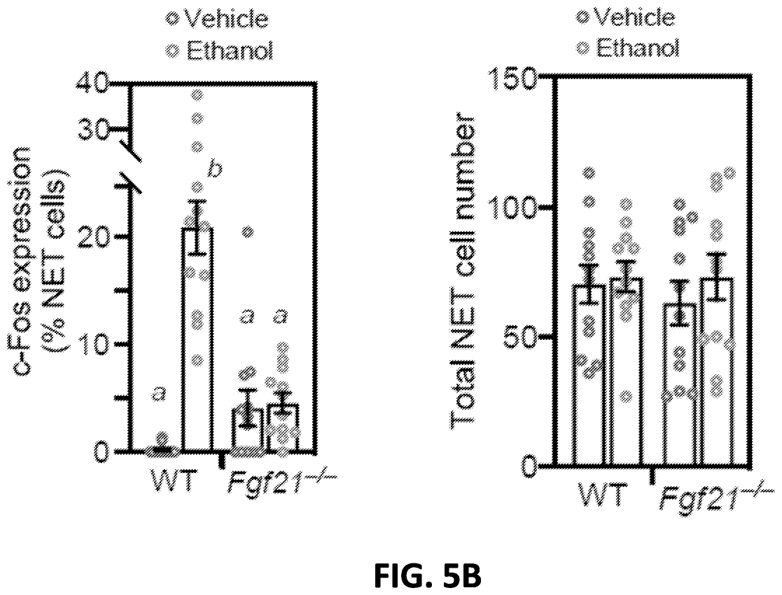

In order to determine whether FGF21 contributes to ethanol-induced activation of noradrenergic neurons in the LC, WT and Fgf21−/− mice were administered ethanol by oral gavage and sacrificed 2.5 hours later. Immunostaining of LC sections was performed for c-Fos and the NE transporter (NET), a marker of noradrenergic neurons. Ethanol induced c-Fos expression in NET+LC neurons of WT mice, as reported (FIG. 5A and FIG. 5B) (Chang et al., 1995; Kolodziejska-Akiyama et al., 2005; Thiele et al., 1997). Remarkably, this inductive response was completely absent in Fgf21−/− mice, although there was a trend towards increased basal c-Fos in the knockout mice (FIG. 5A and FIG. 5B). These data demonstrate that FGF21 is an essential physiologic signal for ethanol to activate noradrenergic neurons in the LC.

### Example 6-FGF21 Acts Directly on Noradrenergic Neurons

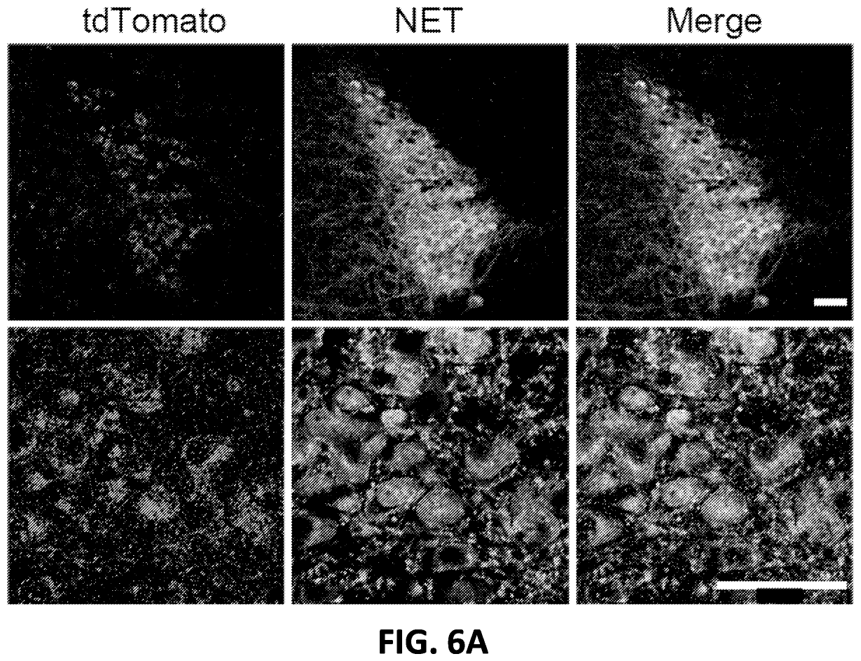

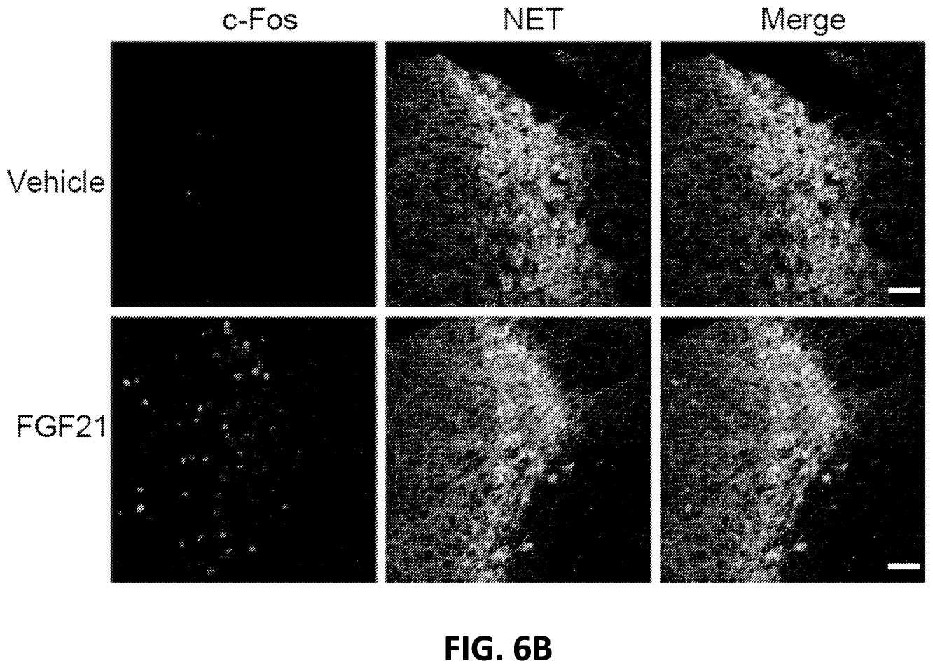

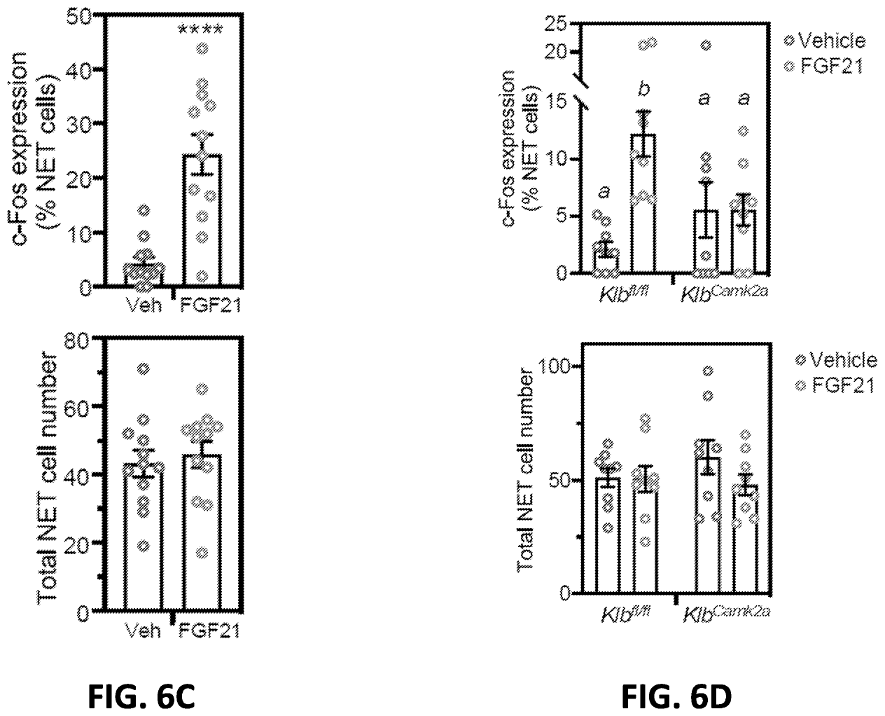

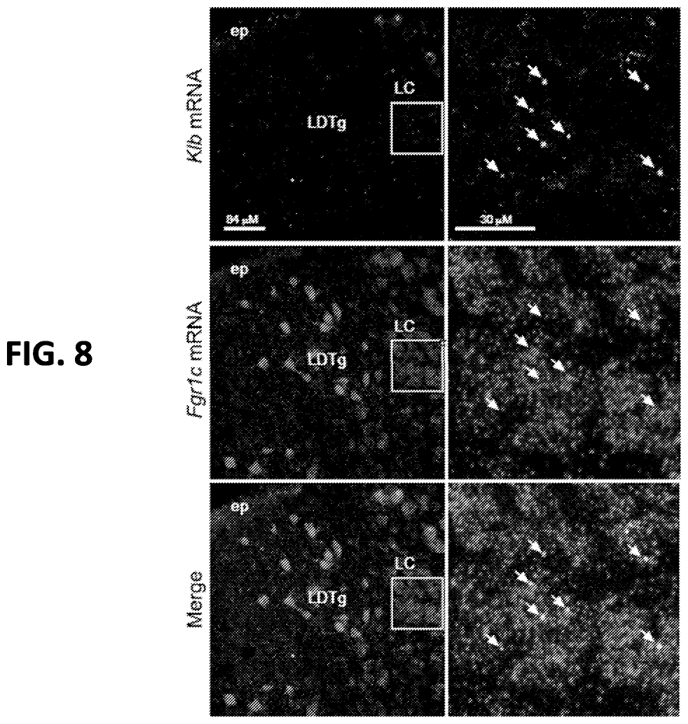

To determine whether FGF21 acts directly on noradrenergic neurons, initial experiments tested whether FGF21's obligate co-receptor, KLB, which is selectively expressed in only a few regions of the brain (Bookout et al., 2013), is present in the LC. Due to the lack of reliable KLB antibodies, KLB expression was measured using an established reporter mouse in which TdTomato was knocked into the endogenous Klb gene, resulting in a KLB-TdTomato fusion protein (Coate et al., 2017). The fusion protein was detected by immunostaining in the LC, where it co-localized with NET in most but not all cells (FIG. 6A). The fusion protein was also detected in regions adjacent to the LC where there was little or no NET staining (FIG. 6A). Thus, KLB is expressed in noradrenergic neurons and possibly other cell types in and around the LC. Due to the lack of FGFR1c-selective antibodies, we examined whether Fgfr1c. mRNA is present in the LC by in situ hybridization. Fgfr1c mRNA was detected throughout the LC region where it colocalized in some cells with Klb mRNA (FIG. 8). In agreement with the Fgfr1c/Klb expression data, pharmacologic FGF21 administration induced c-Fos immunoreactivity in NET+LC neurons of WT mice (FIG. 6B and FIG. 6C). When this same pharmacologic experiment was performed in neuron-specific KlbCamk2a mice, there was no induction by FGF21 and a trend towards increased basal c-Fos expression (FIG. 6D).

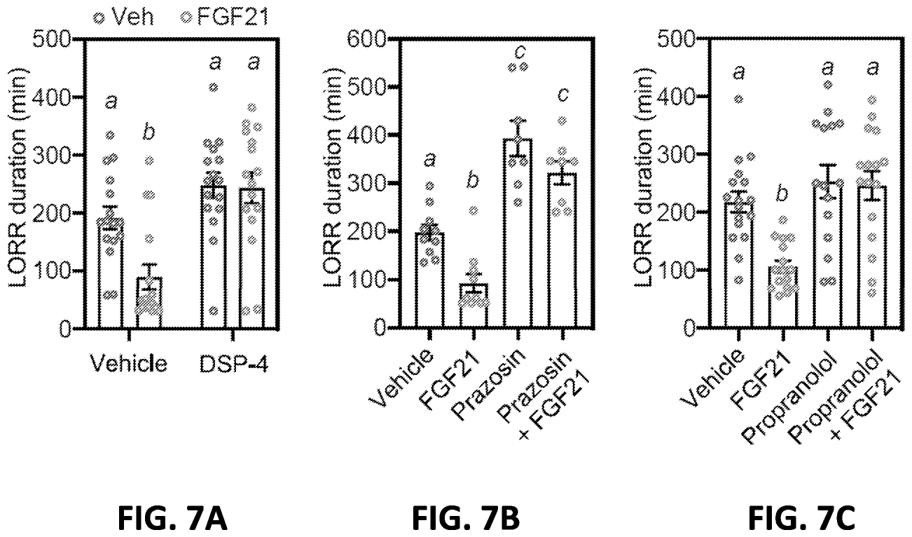

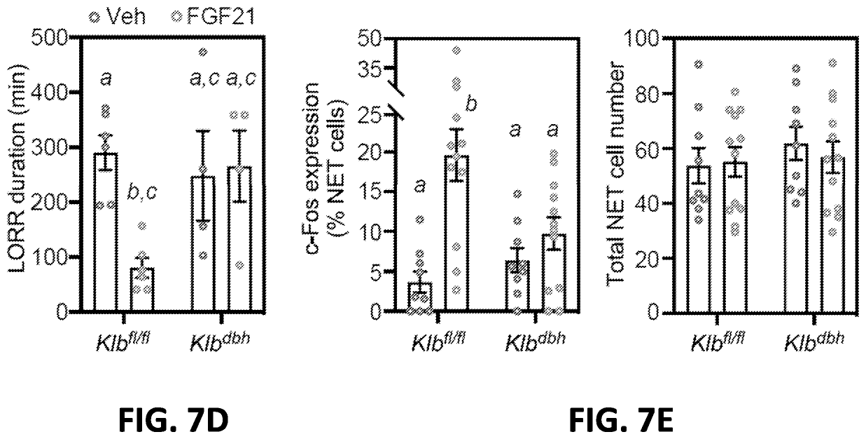

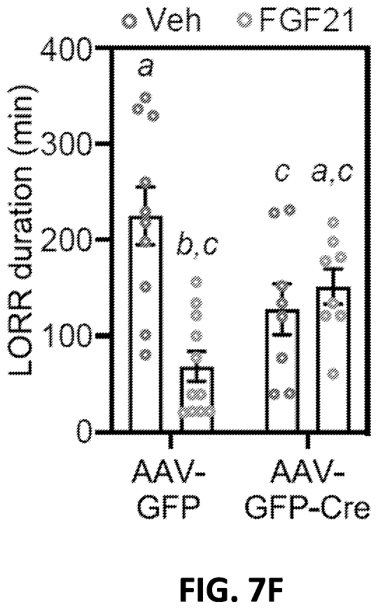

To test whether noradrenergic neurons are required for FGF21's amethystic activity, we first used DSP-4, a neurotoxin that readily crosses the blood-brain barrier and selectively and irreversibly inhibits NET, thereby disrupting NE signaling (Ross and Stenfors, 2015). Mice pretreated with DSP-4 were completely refractory to FGF21's amethystic effect on righting reflex (FIG. 7A). Upon its release from neurons, NE acts on a1-and-adrenergic receptors in multiple subcortical regions to stimulate arousal (Berridge et al., 2012). Accordingly, FGF21's amethystic effect was also blocked by the selective 1-and-adrenergic receptor antagonists, prazosin and propranolol, respectively (FIG. 7B and FIG. 7C). Prazosin on its own prolonged ethanol-induced sedation as previously reported (FIG. 7B) (Malinowska et al., 1999). These pharmacologic data show that FGF21 exerts its amethystic activity through the noradrenergic nervous system.

Lastly, we performed complementary genetic Klb knockout studies to examine whether FGF21 acts directly on neurons that are (i) noradrenergic and (ii) in the LC region. Klbfl/fl mice were either crossed with Dbh-Cre mice to selectively disrupt FGF21 activity in noradrenergic neurons; or they were bilaterally injected in the LC region with adenovirus-associated viruses (AAV) expressing either Cre recombinase fused with green fluorescent protein (GFP) or GFP alone. Consistent with the pharmacologic studies, FGF21's amethystic effect on righting reflex was abolished in KlbDbh mice (FIG. 7D) as well as in Klbfl/fl mice injected with AAV-Cre but not control AAV-GFP (FIG. 7F). Interestingly, unlike Fgf21−/− and KlbCamk2a mice, an increase in the time required for these knockout mice to recover their righting reflex following ethanol exposure was not observed. In Klbfl/fl mice injected with AAV-Cre, the recovery time was even reduced (FIG. 7G). The reason for this difference is not known, but the data raise the possibility of a compensatory response to losing KLB in noradrenergic neurons. In contrast to control mice, FGF21 did not induce c-Fos expression in NET+LC neurons in KlbDbh mice (FIG. 7E). Together these experiments suggest that FGF21 acts directly on neurons that are noradrenergic and in the LC region to exert its amethystic activity.

1. A method of treating acute alcohol intoxication in a subject in need thereof, the method comprising administering an effective amount of an agonist of a fibroblast growth factor receptor (FGFR)/β-klotho complex to the subject.

2. The method of claim 1, wherein treating acute alcohol intoxication comprises reducing disorientation, increasing consciousness, improving heart function, improving respiration, or any combination thereof.

3. The method of claim 1, wherein the agonist comprises FGF21 or an FGF21 congener.

4. The method of claim 1, wherein the effective amount of the agonist is administered as a pharmaceutical composition.

5. The method of claim 1, wherein the effective amount of the agonist is from about 0.5 mg/kg to about 2 mg/kg.

6. The method of claim 1, wherein the subject has imbibed alcohol between about 1 to 24 hours, between about 1 to 18 hours, between about 1 to 12 hours, between about 1 to 10 hours, between about 1 to 8 hours, between about 1 to 6 hours, between about 1 to 5 hours, or between about 1 to 4 hours prior to the administration of the agonist.

7. The method of claim 1, wherein the subject is a human.

8. A pharmaceutical composition comprising an agonist of a fibroblast growth factor receptor (FGFR)/β-klotho complex and a pharmaceutically acceptable carrier or excipient.

9. The pharmaceutical composition of claim 8, wherein the agonist comprises FGF21 or a congener thereof.

10. The pharmaceutical composition of claim 8, wherein the carrier or excipient comprises a diluent, a binder, a filler, a buffering agent, a pH modifying agent, a disintegrant, a dispersant, a preservative, a lubricant or a combination of any thereof.

11. The pharmaceutical composition of claim 8, wherein the composition is formulated for oral, nasal, or intravenous administration.

12. A kit comprising the pharmaceutical composition of claim 8, and instructions for administrating the pharmaceutical composition to a subject suffering from acute alcohol intoxication.

13-17. (canceled)

18. A method of preventing acute alcohol intoxication and/or lessoning the effects of acute alcohol intoxication in a subject in need thereof, the method comprising administering an effective amount of an agonist of a fibroblast growth factor receptor (FGFR)/β-klotho complex to the subject prior to consumption of alcohol by the subject.

19. The method of claim 18, wherein the agonist comprises FGF21 or an FGF21 congener.

20. The method of claim 18, wherein the effective amount of the agonist is administered as a pharmaceutical composition comprising a pharmaceutically appropriate carrier or excipient.

21. The method of claim 18, wherein the effective amount of the agonist is from about 0.5 mg/kg to about 2 mg/kg.

22. The method of claim 18, wherein the subject imbibes alcohol between about 1 to 24 hours, about 1 to 18 hours, about 1 to 12 hours, about 1 to 10 hours, about 1 to 8 hours, about 1 to 6 hours, about 1 to 5 hours or about 1 to 4 hours after to the administration of the FGF21.

23. The method of claim 18, wherein the subject is a human.
